(19) United States
(12) Patent Application Publication
(10) Pub. No.: US 2026/0115431 A1
(43) Pub. Date: Apr. 30, 2026

(54) DELIVERY SYSTEMS FOR CONFORMABLE FILLABLE MEDICAL BALLOONS

(71) Applicants: Boston Scientific Scimed, Inc., Maple Grove, MN (US); Boston Scientific Medical Device Limited, Galway (IE)

(72) Inventors: Benjamin Cleveland, Bellingham, MA (US); Katherine Ann Pollack Knowles, Midvale, UT (US); Cristian Parisi, Amherst, MA (US); Nicolas Ball-Jones, Minneapolis, MN (US); Rick William Dorn, Plymouth, MN (US); Joseph Thomas Delaney, JR., Minneapolis, MN (US); Patrick Nicolas, Mansfield, MA (US); Amit Kumar, Noida (IN); Sushil Kumar, Muzaffarpur (IN); Priyanshu Gupta, Gurgaon (IN); Umesh Kumar Badaya, Bandikui (IN); Jonathan J. Lund, Glencoe, MN (US)

(73) Assignees: Boston Scientific Scimed, Inc., Maple Grove, MN (US); Boston Scientific Medical Device Limited, Galway (IE)

(21) Appl. No.: 19/374,234

(22) Filed: Oct. 30, 2025

Related U.S. Application Data
(60) Provisional application No. 63/714,628, filed on Oct. 31, 2024.
(60) Provisional application No. 63/754,313, filed on Feb. 5, 2025.

(51) Int. Cl. A61M 25/10; A61N 5/10

(52) U.S. Cl. CPC A61M 25/1002; A61M 25/10186; A61N 5/10; A61M 2025/1081; A61N 2005/1094

(57) ABSTRACT

In some aspects, the disclosure provides systems and methods for delivering a conformable, fillable balloon to a target location. A balloon delivery system and balloon may include a tubular elongate shaft with a first lumen extending from a proximal end to a distal end and a hypotube with a second lumen extending from a proximal end to a distal end. A conformable, fillable balloon may be releasably coupled to the distal end of the elongate shaft or hypotube. The balloon may be expandable from a compressed lower profile delivery configuration to an expanded deployed configuration. An interior cavity of the balloon may be in fluid communication with the lumen of the elongate shaft. The balloon is configured to contain a filler material and bound the spread of the filler material as it gels or hardens.

### CROSS-REFERENCE TO RELATED APPLICATIONS

This application claims the benefit of U.S. Provisional Patent Application Ser. No. 63/754,313 filed on Feb. 5, 2025, and U.S. Provisional Patent Application Ser. No. 63/714,628 filed on Oct. 31, 2024, the disclosures of which are incorporated herein by reference in their entireties.

### FIELD

The present disclosure pertains to delivery systems for conformable fillable balloons that are configured to be implanted in a mammalian body for various medical uses.

### BACKGROUND

Various solutions presently exist for spacing, lifting, and embolic applications. However, injectable materials, including in-situ crosslinking materials and shear thinning materials, can have some potential inconveniences that include asymmetric localized deployment of the implanted material, potential for off-target embolization due to delayed reaction or migration of the implanted material, and complexity associated with removal of material if complete removal of the material is desired.

Although these potential inconveniences may be rare, the present disclosure addresses these and other inconveniences by employing conformable, fillable balloons and delivery systems for said balloons to confine injectable materials at the implantation site.

### SUMMARY

The present disclosure is directed to conformable, fillable balloons and that are suitable for implantation in a mammalian body and delivery systems for said balloons.

In an example, a balloon delivery and balloon system may comprise an elongate shaft extending from a proximal end to a distal end, a first lumen extending from the proximal end to the distal end of the elongate shaft, a hypotube extending from a proximal end to a distal end, a second lumen extending from the proximal end to the distal end of the hypotube, and a conformable, fillable balloon releasably coupled to the distal end of the hypotube. The conformable, fillable balloon may define a cavity in fluid communication with the second lumen of the hypotube. The conformable, fillable balloon may be expandable from a compressed lower profile delivery configuration to an expanded deployed configuration. The conformable, fillable balloon may be configured to contain a filler material.

Alternatively or additionally to any of the examples above, in another example, the conformable, fillable balloon may comprise a port at a proximal end thereof.

Alternatively or additionally to any of the examples above, in another example, the port may be disposed within the first lumen of the elongate shaft.

Alternatively or additionally to any of the examples above, in another example, the port may be disposed over and around an outer surface of the hypotube.

Alternatively or additionally to any of the examples above, in another example, the port may comprise a one-way valve.

Alternatively or additionally to any of the examples above, in another example, the conformable, fillable balloon may be compressed such that a maximum lateral width of the balloon is less than an inner diameter of an access sheath.

Alternatively or additionally to any of the examples above, in another example, the conformable, fillable balloon may be compressed by rolling a first lateral side and a second lateral side towards a central axis of the conformable, fillable balloon.

Alternatively or additionally to any of the examples above, in another example, the first lateral side and the second lateral side may be rolled in opposing directions.

Alternatively or additionally to any of the examples above, in another example, the first lateral side and the second lateral side may be rolled in a same direction.

Alternatively or additionally to any of the examples above, in another example, the conformable, fillable balloon may be compressed by folding lateral sides towards a longitudinal axis of the conformable, fillable balloon in an undulating pattern.

Alternatively or additionally to any of the examples above, in another example, the conformable, fillable balloon may be compressed longitudinally such that a compressed length of the conformable, fillable balloon is less than a length of the conformable, fillable balloon in the expanded deployed configuration.

Alternatively or additionally to any of the examples above, in another example, the conformable, fillable balloon may have a longest dimension ranging from 1 millimeter to 40 millimeters when filled.

Alternatively or additionally to any of the examples above, in another example, the conformable, fillable balloon may have a volume ranging from 0.1 milliliters to 250 milliliters when filled.

Alternatively or additionally to any of the examples above, in another example, the conformable, fillable balloon may be formed from a degradable or biodegradable polymeric material.

In an example, a method of implanting a conformable, fillable balloon in a body may comprise inserting a needle to a target location in the body, advancing a dilator over the needle to widen an access path, advancing a sheath over the dilator, removing the dilator while leaving the sheath in place, advancing a balloon delivery system through a lumen of the sheath, a conformable fillable balloon in a compressed configuration releasably coupled to the balloon delivery system, positioning the balloon at the target location, transferring a filler material to an interior cavity of the balloon, causing the balloon to expand, and removing the sheath and the balloon delivery system from the body.

Alternatively or additionally to any of the examples above, in another example, transferring the filler material may comprise injecting the filler material through a lumen of the balloon delivery system.

Alternatively or additionally to any of the examples above, in another example, the method may further comprise imaging the filler material during or after administration using ultrasound or an X-ray-based imaging technique.

Alternatively or additionally to any of the examples above, in another example, the conformable, fillable balloon may be compressed by rolling a first lateral side and a second lateral side towards a central axis of the conformable, fillable balloon.

Alternatively or additionally to any of the examples above, in another example, the conformable, fillable balloon may be compressed by folding lateral sides towards a longitudinal axis of the conformable, fillable balloon in an undulating pattern.

Alternatively or additionally to any of the examples above, in another example, the conformable, fillable balloon may be compressed longitudinally in a folded pattern such that a compressed length of the conformable, fillable balloon is less than a length of the conformable, fillable balloon in an expanded configuration.

In an example, a kit for delivering a conformable, fillable balloon to a target location in a body may comprise an access sheath, a balloon delivery system comprising an elongate shaft extending from a proximal end to a distal end and defining a first lumen extending from the proximal end to the distal end of the elongate shaft, a hypotube extending from a proximal end to a distal end and defining a second lumen extending from the proximal end to the distal end of the hypotube, the hypotube configured to be received in the first lumen of the elongate shaft, a conformable, fillable balloon releasably coupled adjacent to the distal end of the elongate shaft, the conformable, fillable balloon expandable from a compressed lower profile delivery configuration to an expanded deployed configuration. An interior cavity of the balloon may be in fluid communication with the lumen of the elongate shaft. The conformable, fillable balloon may be configured to contain a filler material and bound a spread of the filler material as the filler material gels or hardens.

Alternatively or additionally to any of the examples above, in another example, the conformable, fillable balloon has a series of undulating peaks and valleys when the conformable, fillable balloon is configured in a manufactured configuration.

Alternatively or additionally to any of the examples above, in another example, the peaks and valleys are spaced a distance apart when the conformable, fillable balloon is configured in a manufactured configuration.

Alternatively or additionally to any of the examples above, in another example, the peaks and valleys each have a respective first height when the conformable, fillable balloon is configured in a manufactured configuration.

Alternatively or additionally to any of the examples above, in another example, the conformed, fillable balloon is compressed longitudinally to a compressed configuration such that a compressed length of the conformable, fillable balloon in the compressed configuration is less than a length of the conformable, fillable balloon in the expanded deployed configuration.

Alternatively or additionally to any of the examples above, in another example, the conformable, fillable balloon is compressed longitudinally by translating a first portion of the conformable, fillable balloon including a first valley located on the distal end of the conformable, fillable balloon longitudinally toward a second portion of the conformable, fillable balloon including a second valley.

Alternatively or additionally to any of the examples above, in another example, the conformable, fillable balloon configured in the compressed configuration has a substantially circular shape.

Alternatively or additionally to any of the examples above, in another example, the peaks and valleys of the conformable, fillable balloon configured in the compressed configuration have respective second heights that are greater than the first height.

Alternatively or additionally to any of the examples above, in another example, the longitudinally compressed conformable, fillable balloon with the substantially circular shape is configured to form a substantially rectangular shape when the conformable, fillable balloon is configured in the expanded deployed configuration.

Alternatively or additionally to any of the examples above, in another example, the conformable, fillable balloon has a longest dimension ranging from 1 millimeter to 40 millimeters when filled; and the conformable, fillable balloon has a volume ranging from 0.1 milliliters to 250 milliliters when filled.

Alternatively or additionally to any of the examples above, in another example, a maximum lateral dimension of the conformable, fillable balloon in the compressed lower profile delivery configuration may be less than an inner diameter of the access sheath.

The above summary of some embodiments is not intended to describe each disclosed embodiment or every implementation of the present disclosure. The Figures, and Detailed Description, which follow, more particularly exemplify some of these embodiments.

### BRIEF DESCRIPTION OF THE DRAWINGS

FIG. 1 is a partial cross-section view illustrating an example filler, in the form of a gel composition having been delivered between a rectum and a prostate of a patient in the Denonvilliers' space;

FIG. 2 is a perspective view of a distal end region of an illustrative balloon delivery catheter for delivering a conformable fillable balloon;

FIG. 3A is an end view of the illustrative balloon having an alternative collapsed configuration;

FIG. 3B is another end view of the illustrative balloon having an alternative collapsed configuration;

FIGS. 4A-4F schematically illustrate a method of implanting the balloon in a subject;

FIG. 5 is a side view of another of a distal end region of another illustrative balloon delivery catheter for delivering a conformable fillable balloon; and

FIGS. 6A-6C schematically illustrate portions of a method of implanting the balloon of FIG. 5 in a subject;

FIGS. 7A-7C illustrate side views of another example of a conformable fillable balloon configured in a manufactured configuration, a folded state, and an inflated state, respectively; and

FIGS. 8A-8F schematically illustrate a method of implanting the balloon in a subject.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

The present disclosure is directed to systems and method for delivering a conformable, fillable balloon suitable for implantation in a mammalian body.

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or”unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings. The drawings, which are not necessarily to scale, depict illustrative embodiments and are not intended to limit the scope of the disclosure.

Various solutions presently exist for spacing, lifting, and embolic applications. However, injectable materials, including in-situ crosslinking materials and shear thinning materials, can have some potential inconveniences that include asymmetric localized deployment of the implanted material, potential for off-target embolization due to delayed reaction or migration of the implanted material, and complexity associated with removal of material if complete removal of the material is desired. In one illustrative example, injectable materials may be used between the rectum and the prostate to prevent damage during radiation therapy. One illustrative system for injecting materials into the space between the rectum and the prostate is the SpaceOAR Vue™ System available from Boston Scientific Corporation, Marlborough, MA, USA. In the SpaceOAR Vue™ System, the product may be in a liquid form before it sets into a solid/gel implant. The flowable pre-gel may be somewhat restricted in the superior direction (e.g., by the seminal vesicles and associated structures) and in the inferior direction (e.g., by the Denonvilliers' fascia). However, there may be no anatomy present which limits or prevents lateral (e.g., side to side) flow of the pre-gel. Laterally, the pre-gel may flow into appendages or off-shoots outside of the target location which results in the implant setting in a suboptimal shape. Further, some spacing hydrogels may experience hydrogel migration or may not be localized at the implant site for an entirety of a procedure. It is further contemplated that the risk of uncontrolled or partially uncontrolled placement at a target site may preclude the use of materials which experience slow or partially uncontrolled cross-linking reactions or at risk for off-target polymerization and/or migration. The present disclosure is directed towards devices to limit a flow of the pre-gel and methods and systems for delivering said devices.

While the present disclosure is described with respect to injecting materials between the rectum and the prostate to prevent damage during radiation therapy, the systems and methods described herein may be used in other anatomies, as desired. The systems and methods described herein may be useful for a number of medical procedures including spacing, lifting, bulking, embolization and backfill procedures. Spacing procedures include soft tissue spacing procedures wherein one or more fillable balloons are placed, for example, between the rectum and the prostate and filled to prevent damage during radiation therapy or between other organs and/or tissues to protect them from potential side effects of a particular treatment, and joint spacing procedures where one or more fillable balloons are placed in the space of a joint, such as a shoulder, hip, knee or ankle joint and filled to provide mechanical support for the joint. Lifting procedures include procedures where one or more fillable balloons are placed and filled to lift a sinus membrane for sinus augmentation or others. Bulking procedures include procedures wherein one or more fillable balloons are implanted in or adjacent to a bodily sphincter, such as an anal sphincter or a urethral sphincter, to address intrinsic sphincter deficiency or others. Embolization procedures include those where one or more fillable balloons are placed in a target blood vessel and filled to block blood flow to an area of the body. Backfill procedures include procedures wherein one or more fillable balloons are implanted and filled in a left atrial appendage after the introduction of a closure device such as the Watchman® left atrial appendage closure device available from Boston Scientific Corporation, Marlborough, MA, USA or the balloon is used as a closure device for the left atrial appendage with no additional support or devices.

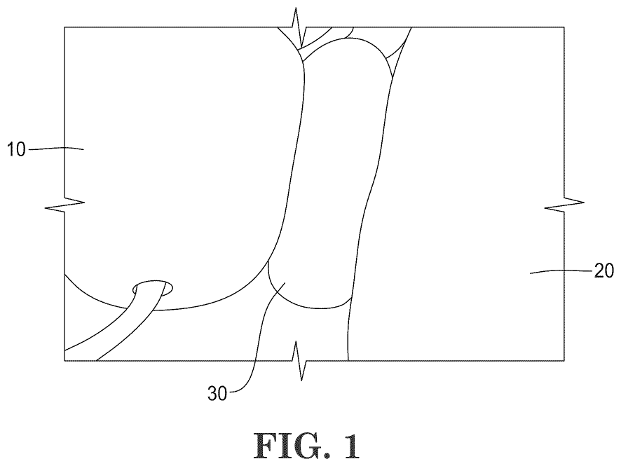

Turning to the drawings, FIG. 1 is a partial cross-section view illustrating an example conformable, fillable balloon 30, having been delivered and filled between a rectum 20 and a prostate 10 of a patient in the Denonvilliers' space. The balloon 30 may be a conformable, fillable balloon such as those described herein.

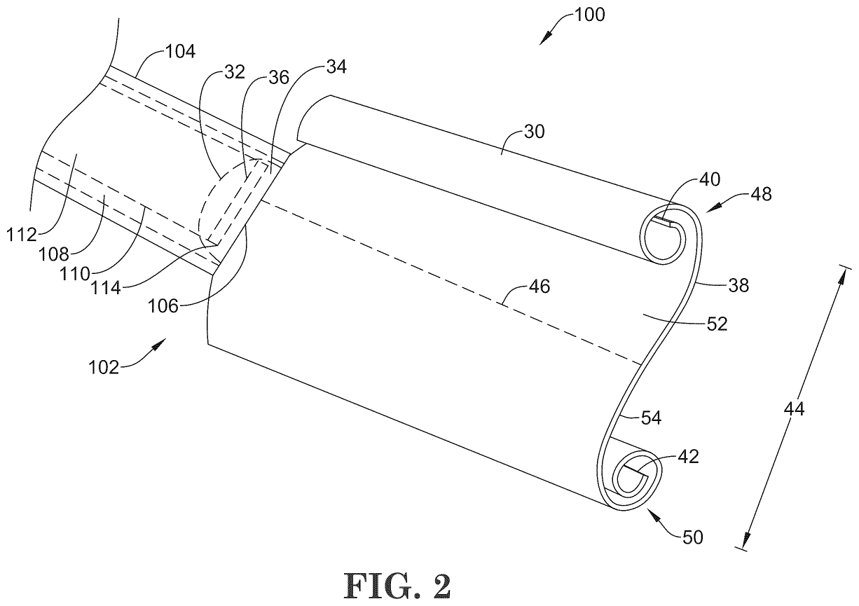

FIG. 2 is a perspective view of a distal end region 102 of an illustrative balloon delivery system 100 for delivering a conformable fillable balloon 30. The balloon 30 may be expandable from a compressed lower profile delivery configuration to an expanded deployed configuration. The balloon delivery system 100 may include a tubular elongate shaft 104 extending from a proximal end (not explicitly shown) configured to remain outside the body to a distal end 106. A shaft lumen 108 may extend from the proximal end of the elongate shaft 104 to the distal end 106 thereof. The proximal end of the shaft lumen 108 may be configured to receive a hypotube 110, or other tubular member, therethrough. The hypotube 110 may include a lumen 112 extending from a proximal end to a distal end 114 thereof. The proximal end of the hypotube 110 may be configured to be coupled to a filler material source and a filler material may be injected into the conformable, fillable balloon 30 through the lumen 112. For example, the proximal end of the hypotube lumen 112 may be in fluid communication with a filler injection system. An interior cavity of the balloon 30 may be in fluid communication with the lumen 112 of the hypotube 110 to receive the filler material therein. In some embodiments, the hypotube 110 may be omitted and the filler material may be injected into the balloon 30 via the shaft lumen 108 of the elongate shaft 104.

A proximal end 32 of the balloon 30 may be releasably coupled or secured to the distal end 114 of the hypotube 110. In some cases, the proximal end 32 of the balloon 30 may include a port 34 that is configured to be received within the lumen 112 of the hypotube 110. In other embodiments, the port 34 may be disposed over and around an outer surface of the hypotube 110. In embodiments where the hypotube 110 is omitted, the proximal end 32 of the balloon 30 may be releasably coupled or secured to the distal end 106 of the elongate shaft 104 either within the shaft lumen 108 thereof or over and around an outer surface of the elongate shaft 104. While coupling of the balloon 30 relative to the system 100 is described with the hypotube 110 present, in the absence of the hypotube 110, the balloon 30 may be coupled to the elongate shaft 104 in similar manners. Further, delivery of the filler material may occur through the lumen 108 of the elongate shaft 104 in a similar manner to that described with respect to the hypotube 110.

The port 34 is releasably coupled to the hypotube 110 such that the interior of the balloon 30 is in fluid communication with the lumen 112 of the hypotube 110. The port 34 may include a one-way or a check valve 36, such as, but not limited to, a duckbill valve that allows flow of filler material into the balloon 30 while preventing reverse flow of filler material out of the balloon 30. In some cases, the distal end 114 of the hypotube 110 may extend distally beyond the valve 36. However, this is not needed. As another example, the balloon 30 may be provided with an aperture which allows flow of filler material into the balloon and a plug that is configured to seal the aperture to prevent flow of filler material out of the balloon 30 once filled. In yet another example, the balloon 30 may be provided with an aperture through which a small tube such as a hypotube is inserted for filling the balloon 30, which aperture is resealed upon withdrawal of the tube from the aperture (e.g., due to elastic recovery of the aperture or a constricting device that constricts the aperture, such as a clamp or elastic ring), preventing flow of filler material out of the balloon 30.

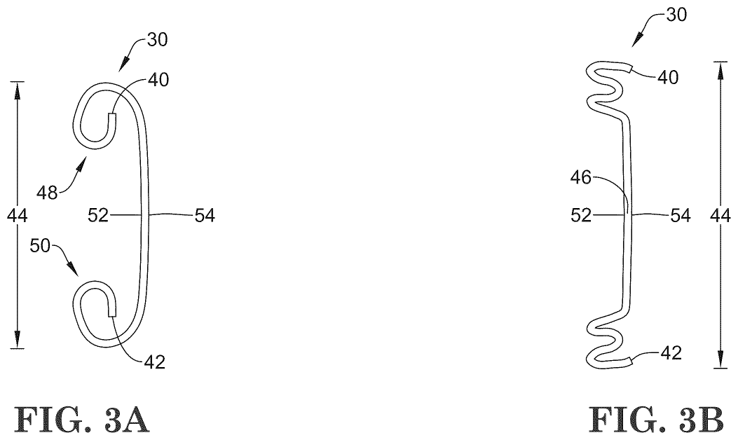

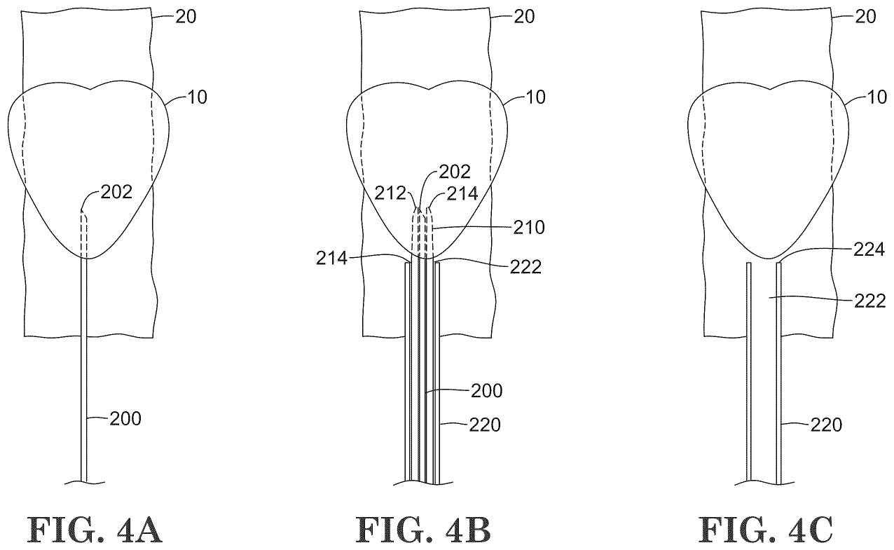

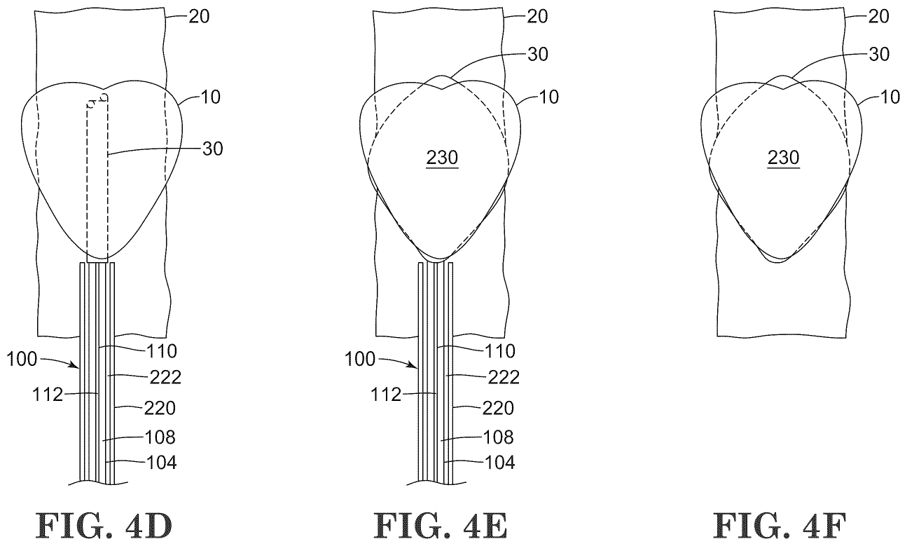

The balloon 30 may extend distally from the proximal end 32 to a distal end 38. The balloon 30 may extend laterally from a first lateral side 40 to a second lateral side 42. In FIG. 2, the balloon 30 has been compressed into a delivery configuration having a reduced profile. The balloon 30 may be compressed such that a maximum lateral width 44 of the balloon 30 is less than the inner diameter of an access sheath (for example, sheath 220 in FIGS. 4B-4E). This may allow the balloon delivery system 100 to be advanced through the lumen of the access sheath. It is contemplated that the balloon 30 may be compressed in a manner that allows for relatively even expansion in the lateral directions. For example, in the illustrated embodiment, the first lateral side 40 of the balloon 30 may be rolled towards a central axis 46 of the balloon 30 to define a first rolled portion 48. Similarly, the second lateral side 42 of the balloon 30 may also be rolled towards the central axis 46 to create a second rolled portion 50. When the balloon delivery system 100 is used to deliver the balloon 30 to the space between the rectum and the prostate, the axes of the first and second rolled portions 48, 50 may extend generally in the superior/inferior direction. It is contemplated that the first and second rolled portions 48, 50 may be rolled in opposing directions (as shown in FIG. 2). For example, the first rolled portion 48 may be rolled along a first surface 52 while the second rolled portion 50 may be rolled along a second surface 54. The second surface 54 may face in a direction away from the first surface 52. Referring briefly to FIG. 3A, which illustrates an end view of the illustrative balloon 30 having an alternative collapsed configuration, in some configurations, the first and second rolled portions 48, 50 may be rolled in a same direction. For example, the first and second rolled portions 48, 50 may be rolled along a same surface 52 of the balloon 30. It is contemplated that the first and second rolled portions 48, 50 may be rolled along the first surface 52 or the second surface 54, as desired. It is further contemplated that how far the first and second rolled portions 48, 50 are rolled towards the central longitudinal axis 46 of the balloon 30 may be varied. In some cases, one or both of the rolled portions 48, 50 may be rolled until a respective edge thereof is at or adjacent to the central longitudinal axis 46 such that most or all of the balloon 30 is rolled between the width 44 thereof. However, this is not needed. In some cases, there may be a portion (e.g., a central region) of the balloon 30 that is not rolled, as shown in FIGS. 2 and 3A.

It is contemplated that the balloon 30 may be equally compressed in the lateral direction to provide more uniform and predictable inflation of the balloon 30 in the body. For example, if the balloon delivery system 100 is delivered centrally to the desired deployment location, the lateral sides 40, 42 may expand generally uniformly in opposing lateral directions to centrally deploy the balloon 30. It is contemplated that the balloon 30 may be compressed using other compression techniques. For example, referring additionally to FIG. 3B, which illustrates an end view of the illustrative balloon 30 having another alternative collapsed configuration, the lateral sides 40, 42 may be folded or crimped towards the longitudinal axis 46. The lateral edges may be folded back and forth on themselves in an undulating or accordion pattern to reduce the lateral width 44 of the balloon 30. The folds may be positioned along an axis generally orthogonal to the longitudinal axis 46 of the balloon 30. It is contemplated that the lateral sides 40, 42 may be rolled, folded, compressed, in any number of different ways or combinations of ways towards the central longitudinal axis 46 to reduce the lateral width 44 thereof. Further, in some cases, a longitudinal length of the balloon 30 may be additionally or alternatively compressed or shortened for the delivery configuration. It is contemplated that the material from which the balloon 30 is constructed may influence how the balloon 30 is compressed.

FIGS. 4A-4F schematically illustrate a method of implanting the balloon 30 in a subject. The illustrative method is shown and described with respect to implantation between the prostate 10 and the rectum 20. However, the balloon 30 may be delivered to other anatomies using similar delivery systems and methods. While certain steps are shown as a sequence between each figure, in other embodiments fewer steps are contemplated and the order by which steps are performed can be different than what is illustrated. In some embodiments, the methods herein can comprise puncturing the intended anatomy using a dilator and advancing the dilator and sheath assembly inside the anatomy until the sheath is at an intended location in vivo (e.g., under prostate). Optionally, the methods herein can include performing dissection of tissue (e.g., hydro dissection as needed by injecting a fluid (e.g., saline) through a luer provided on the proximal end of the dilator), retracting the dilator from the sheath, inserting and advancing a balloon assembly in vivo until the balloon reaches a distal end of the sheath. The methods herein can include retracting the sheath to expose a folded balloon, such as those described herein. The methods herein can include injecting a filler (e.g., hydrogel) until the balloon is configured in an inflated configuration (e.g., completely inflated), pulling a balloon catheter while keeping the sheath pressed against the inflated balloon until the balloon catheter assembly is detached from balloon, and/or retracting the sheath and the balloon catheter assembly from the anatomy, as described herein.

For instance, as shown in FIG. 4A, a needle 200 may be inserted to the target location. In the illustrated example, the target location may be between the prostate 10 and the rectum 20. The target location may be centrally located in the lateral direction. The needle 200 may be an 18-gauge hypodermic needle. However, the needle 200 may be smaller than 18 gauge or greater than 18-gauge, as desired. Exemplary materials for the needle include, but are not limited to, metals and metal alloys, such as stainless steel and Nitinol, and polymers. The distal tip 202 of the needle may be sharpened and may have a beveled shape. Next, a dilator 210 may be advanced over the needle 200 to widen the access path, as shown in FIG. 4B. For example, the dilator 210 may include a lumen 212 extending from a proximal end (not shown) to a distal end 214 of the dilator 210. The lumen 212 may be sized and shaped to receive the needle 200 therein. A sheath 220 may be advanced over the dilator 210. In some cases, the sheath 220 may be advanced substantially simultaneously with the dilator 210. In other cases, the access sheath 220 may be advanced over the dilator 210 after the dilator 210 has been positioned. The sheath 220 may include a lumen 222 extending from a proximal end (not shown) to a distal end 224 of the sheath 220. The lumen 222 may be sized and shaped to receive the dilator 210 and the balloon delivery system 100 therein. In some examples, the lumen 222 may have diameter of approximately 10 millimeters (mm). However, the diameter of the lumen 222 may be less than 10 mm or greater than 10 mm.

The dilator 210 may then be removed while the sheath 220 is left in place, as shown in FIG. 4C. Next the balloon delivery system 100 may be advanced through the lumen 222 of the sheath 220 with the balloon 30 in the compressed or compact configuration. The balloon delivery system 100 may be distally advanced until the balloon 30 extends distally beyond the distal end 224 of the sheath 220 and is positioned at the target location, as shown in FIG. 4D. The filler material 230 may then be transferred to the interior cavity of the balloon 30. In some cases, the filler material 230 may be injected through the lumen 112 of the hypotube 110 in a flowable state. The port 34 of the balloon 30 may be fluidly coupled to the lumen 112 to allow the filler material 230 to be injected into the cavity of the balloon 30. As the filler material 230 fills the cavity, the balloon 30 expands in lateral directions away from the central axis 46 of the balloon 30. The balloon 30 contains the filler material and bounds the spread of the filler material 230 as the filler material 230 gels or hardens, as shown in FIG. 4E. In some embodiments, the filler material 230 of the present disclosure can be imaged during or after administration using a suitable imaging technique such as ultrasound or an X-ray-based imaging technique, such as computerized tomography or X-ray fluoroscopy.

Once the balloon 30 is full, the sheath 220 and the balloon delivery system 100 may be removed from the body, as shown in FIG. 4F. It is contemplated that the balloon 30 may be automatically disengaged from the balloon delivery system 100 once the balloon 30 is full. In other embodiments, the sheath 220 may be distally advanced to push the balloon 30 off of the balloon delivery system 100. In yet other examples, the balloon delivery system 100 may be proximally retracted prior to proximal retraction of the sheath 220. The distal end 224 of the sheath 220 may act as a mechanical stop to disengage the balloon 30 from the balloon delivery system 100. The balloon delivery system 100 and the sheath 220 may be proximally retracted simultaneously, one after the other, or combinations thereof.

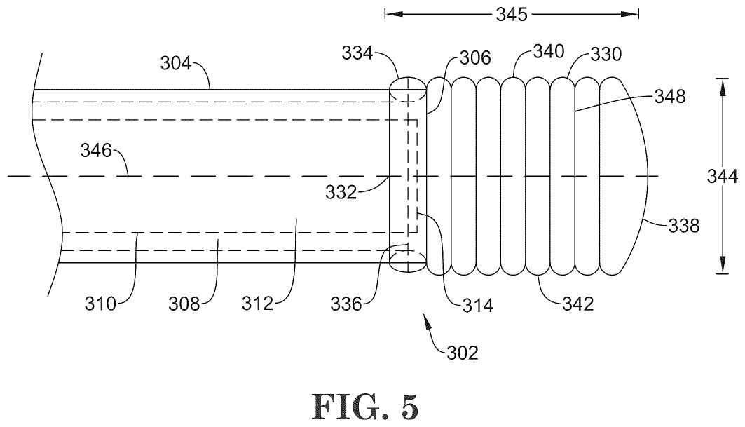

FIG. 5 is a side view of a distal end region 302 of another illustrative balloon delivery system 300 for delivering a conformable fillable balloon 330. The balloon 330 may be expandable from a compressed lower profile delivery configuration to an expanded deployed configuration. The balloon delivery system 300 may include a tubular elongate shaft 304 extending from a proximal end (not explicitly shown) configured to remain outside the body to a distal end 306. A lumen 308 may extend from the proximal end of the elongate shaft 304 to the distal end 306 thereof. The proximal end of the shaft lumen 308 may be configured to receive a hypotube 310, or other tubular member, therethrough. The hypotube 310 may include a lumen 312 extending from a proximal end to a distal end 314 thereof. The proximal end of the hypotube 310 may be configured to be coupled to a filler material source and a filler material may be injected into the conformable, fillable balloon 330. For example, the proximal end of the hypotube lumen 312 may be in fluid communication with a filler injection system. An interior cavity of the balloon 330 may be in fluid communication with the lumen 312 of the hypotube 310 to receive the filler material therein. In some embodiments, the hypotube 310 may be omitted and the filler material may be injected into the balloon 330 via the lumen 308 of the elongate shaft 304.

A proximal end 332 of the balloon 330 may be releasably coupled or secured to the distal end 306 of the elongate shaft 304. In some cases, the proximal end 332 of the balloon 330 may include a port 334 that is configured to be disposed over and around an outer surface of the elongate shaft 304. In other embodiments, the port 334 may be disposed within the lumen 308 of the elongate shaft 304. The port 334 is releasably coupled to the elongate shaft 304 such that the interior of the balloon 330 is in fluid communication with the lumen 312 of the hypotube 310. In other embodiments, the balloon 330 may be releasably coupled within the hypotube lumen 312 or over and around an outer surface of the hypotube 310. The port 334 may include a one-way or a check valve 336, such as, but not limited to, a duckbill valve that allows flow of filler material into the balloon 330 while preventing reverse flow of filler material out of the balloon 330. As another example, the balloon 330 may be provided with an aperture which allows flow of filler material into the balloon 330 and a plug that is configured to seal the aperture to prevent flow of filler material out of the balloon 330 once filled. In yet another example, the balloon 330 may be provided with an aperture through which a small tube such as a hypotube is inserted for filling the balloon 330, which aperture is resealed upon withdrawal of the tube from the aperture (e.g., due to elastic recovery of the aperture or a constricting device that constricts the aperture, such as a clamp or elastic ring), preventing flow of filler material out of the balloon 330.

The balloon 330 may extend distally from the proximal end 332 to a distal end 338. The balloon 330 may extend laterally from a first lateral side 340 to a second lateral side 342. In FIG. 5, the balloon 330 has been compressed into a delivery configuration having a reduced profile. The balloon 330 may be compressed such that a maximum lateral width 344 of the balloon 330 is less than the inner diameter of an access sheath (for example, sheath 220). This may allow the balloon delivery system 300 to be advanced through the lumen of the access sheath. Further, the balloon 330 may be compressed longitudinally (e.g., parallel to the longitudinal axis 346 thereof) such that the compressed longitudinal length 345 of the balloon 330 is less than a longitudinal length of the balloon 330 in the expanded configuration. It is contemplated that the balloon 330 may be compressed in a manner that allows for relatively even expansion in the lateral directions. For example, in the illustrated embodiment, the distal end 338 of the balloon 330 may be compressed towards the proximal end 332. As the distal end 338 of the balloon 330 is compressed towards the proximal end 332, the first and second lateral sides 340, 342 may be tucked inwards towards the central longitudinal axis 346 in a back-and-forth manner (e.g., like an accordion) to form a plurality of folds 348. The folds 348 may extend or stack in a direction generally parallel to the longitudinal axis 346 of the balloon 330. It is contemplated that the balloon 330 may be equally compressed in the lateral direction to provide more uniform and predictable inflation of the balloon 330 in the body. For example, if the balloon delivery system 300 is delivered centrally to the desired deployment location, the lateral sides 340, 342 may expand generally uniformly in opposing lateral directions to centrally deploy the balloon 330. It is contemplated that the balloon 330 may be compressed using other compression techniques.

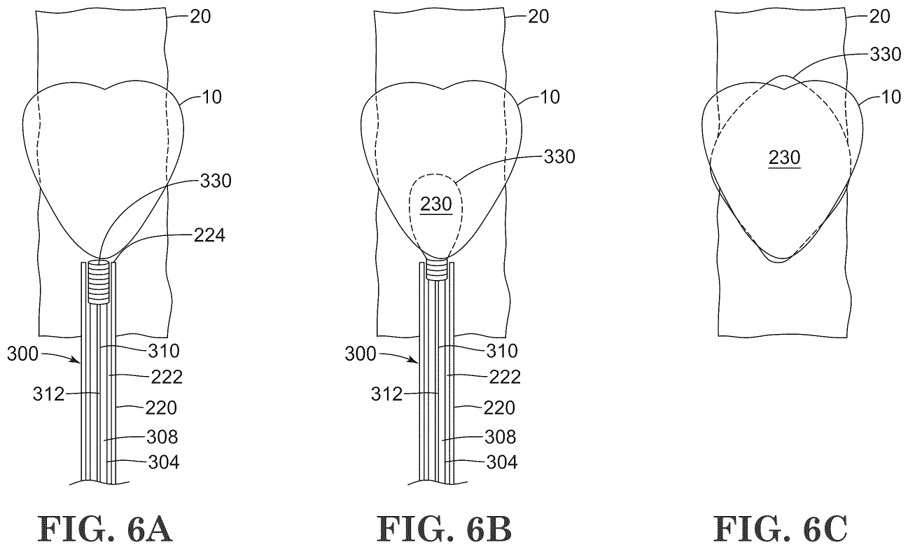

FIGS. 6A-6C schematically illustrate portions of a method of implanting the balloon 330 in a subject. The illustrative method is shown and described with respect to implantation between the prostate 10 and the rectum 20. However, the balloon 330 may be delivered to other anatomies using similar delivery systems and methods. While certain steps are shown as a sequence between each figure, in other embodiments fewer steps are contemplated and the order by which steps are performed can be different than what is illustrated. The first steps of the procedure may be similar to the steps described with respect to FIGS. 4A-4C and have not been duplicated. Generally, a needle 200 may be inserted to the target location (FIG. 4A). Next, a dilator 210 may be advanced over the needle 200 to widen the access path (FIG. 4B). A sheath 220 may be advanced over the dilator 210. The dilator 210 may then be removed while the sheath 220 is left in place (FIG. 4C).

Next the balloon delivery system 300 may be advanced through the lumen 222 of the sheath 220 with the balloon 330 in the compressed or compact configuration. The balloon delivery system 300 may be distally advanced until the balloon 330 is adjacent to the distal end 224 of the sheath 220, as shown in FIG. 6A. However, this is not required. In some embodiments, the balloon delivery system 300 may be distally advanced until the balloon 330 extends distally beyond the distal end 224 of the sheath 220 and is positioned at the target location. The filler material 230 may then be transferred to the interior cavity of the balloon 330. In some cases, the filler material 230 may be injected through the lumen 312 of the hypotube in a flowable state. The port 334 of the balloon 330 may be fluidly coupled to the shaft lumen 308 to allow the filler material 230 to be injected into the cavity of the balloon 330. As the filler material 230 fills the cavity, the balloon 330 expands in a distal direction and in lateral directions away from the central axis 346 of the balloon 330, as shown in FIG. 6B. The balloon 330 contains the filler material and bounds the spread of the filler material 230 as the filler material 230 gels or hardens. In some embodiments, the filler material 230 of the present disclosure can be imaged during or after administration using a suitable imaging technique such as ultrasound or an X-ray-based imaging technique, such as computerized tomography or X-ray fluoroscopy.

Once the balloon 330 is full, the sheath 220 and the balloon delivery system 300 may be removed from the body, as shown in FIG. 6C. It is contemplated that the balloon 330 may be automatically disengaged from the balloon delivery system 300 once the balloon 330 is full. In other embodiments, the sheath 220 may be distally advanced to push the balloon 330 off of the balloon delivery system 300. In yet other examples, the balloon delivery system 300 may be proximally retracted prior to proximal retraction of the sheath 220. The distal end 224 of the sheath 220 may act as a mechanical stop to disengage the balloon 330 from the balloon delivery system 300. The balloon delivery system 300 and the sheath 220 may be proximally retracted simultaneously, one after the other, or combinations thereof.

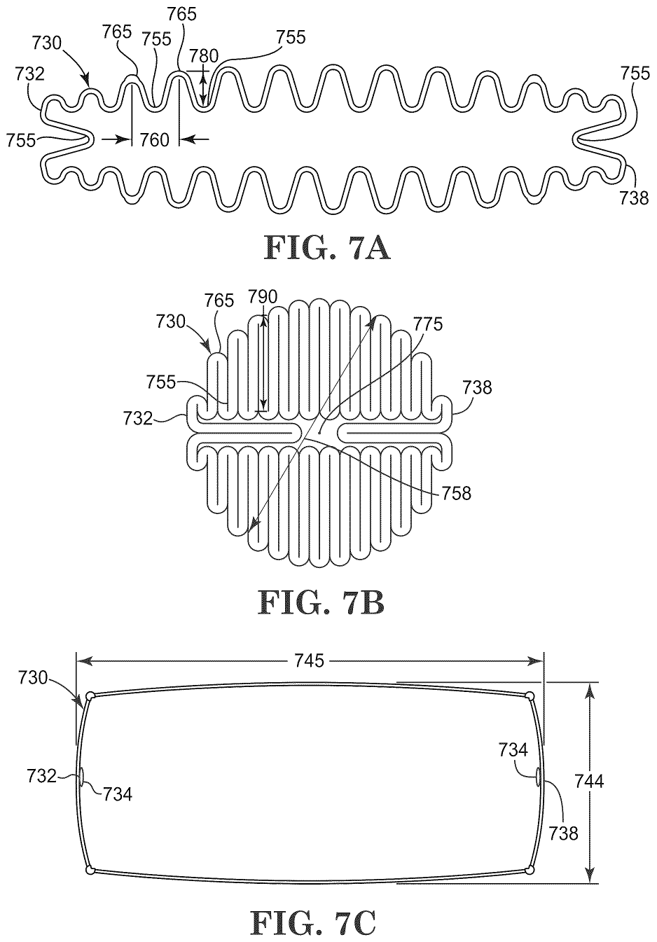

FIGS. 7A-7C illustrate side views of another example of a conformable fillable balloon 730. The balloon 730 can be deployed in vivo using any of the above-mentioned balloon delivery systems e.g., the balloon delivery systems 100, 300 but with the presence of the conformable fillable balloon 730 instead of the other types of a conformable fillable balloons as described herein. The balloon 730 can have a substantially uniform thickness or width as taken at any point along the balloon 730 (e.g., at any point along the peaks 765 and valleys 755) when the balloon is in the manufactured configuration, the folded delivery state, and the deployed expanded state.

FIG. 7A illustrates the conformable fillable balloon 730 in a manufactured configuration (i.e., an as manufactured configuration or an initial configuration). The conformable fillable balloon 730 is manufactured with an undulating wave pattern with peaks and valleys along its entire length. This manufactured configuration is specifically designed to facilitate controlled folding and optimal deployment of the conformable fillable balloon 730 in vivo. That is, the conformable fillable balloon 730 is configured in a non-compressed (e.g., non-longitudinally compressed) manufactured configuration in which the conformable, fillable balloon 730 has a substantially rectangular shape formed of an undulating series of peaks 765 and valleys 755 extending along the entire perimeter of the balloon 730. The peaks 765 and valleys 755 can each have a respective height when the balloon 730 is configured in the manufactured configuration. The peaks and valleys 755 can each be spaced a distance 760 apart (e.g., longitudinally apart) when the balloon 730 is configured in the manufactured configuration. Hence, the peaks 765 and valleys 755 can have a first height 780 and the peaks 765 and the valleys 755 can be spaced the distance 760 from adjacent peaks 765 and valleys 755 when the balloon 730 is configured in the manufactured configuration. As illustrated in FIG. 7A, valleys located proximal to or at the distal end 738 and the proximal end 732 of the balloon 730 can be extend substantially longitudinally in opposing directions (e.g., with a first valley at the distal end 738 extending proximally inward toward a center of the balloon 730 and with the second valley at the proximal end 732 extending distally inward toward the center of the balloon 730) when the balloon 730 is configured in the manufactured configuration. Similarly, the peaks and valleys located on opposing surfaces of the balloon 730 that extend between the proximal and distal ends 732, 738 of the balloon 730 can extend substantially radially in opposing directions, as illustrated in FIG. 7A.

FIG. 7B illustrates the conformable fillable balloon 730 in a compressed configuration, namely a circular folded pattern. Stated differently, the conformable fillable balloon 730 has a substantially circular shape when the conformable fillable balloon 730 is configured in the compressed folded configuration. Having a circular shape when in conformable fillable balloon 730 is in the compressed configuration can promote aspects herein such as permitting the compressed conformable fillable balloon 730 to be readily disposed within a lumen of a component included in a balloon delivery system. When the balloon 730 is configured in the compressed configuration a compressed length of the conformable, fillable balloon 730 is less than a longitudinal length of the conformable, fillable balloon 730 in the expanded deployed configuration, as described herein.

The compression of the balloon 730 can be performed by compressing the balloon 730 longitudinally. For instance, opposing sides of the conformable, fillable balloon 730 can be compressed (e.g., moved toward each other) such that the series of undulating peaks 765 and the valleys 755 are compressed (e.g., longitudinally) and thus expand radially to a second height 790 that is greater than the first height (e.g., the first height 780 as illustrated in FIG. 7A). For example, the valley located at the proximal end 732 of the balloon 730 can be moved toward the valley located at the distal end 738 of the balloon to until each of the peaks and valleys located along the opposing surfaces of the balloon extending between the proximal and distal ends 732, 738 are in contact with adjacent peaks and/or valley, as illustrated in FIG. 7B. For instance, each of the peaks 765 and valleys 755 can have a respective second height 790 when the balloon 730 is configured in the compressed configuration that is greater than a respective first height of the peaks 765 and valleys 755 when the balloon 730 is configured in the manufactured configuration. The peaks 765 and valleys 755 can each be in contact with one or more adjacent peaks 765 and/or one or more valleys 755 when the balloon 730 is configured in the folded configuration, as illustrated in FIG. 7B. Hence, the peaks 765 and valleys 755 can have a second height and the peaks 765 and the valleys 755 can be in contact with one or more adjacent peaks 765 and/or valleys 755 (e.g., are no longer spaced the distance 760 from adjacent peaks 765 and valleys 755), thereby yielding the overall substantially circular shape of the balloon 730 in the compressed configuration. The compression (folding) of the balloon 730 creates a compact profile while maintaining structural integrity for deployment. The folded configuration achieves the lowest possible profile (<9 F) while ensuring consistent expansion of the balloon 730.

In some embodiments, the balloon 730 can have a circumference 758 in a range from about 1 millimeter to about 5 millimeters when the balloon 730 is configured in the compressed (delivery) configuration. For instance, the balloon can have a radial circumference 758 in a range from about 1 millimeter to about 5 millimeters when the balloon 730 is configured in the compressed configuration. For example, the balloon 730 can have a circumference 758 of about 2 millimeters, about 3 millimeters, about 4 millimeters, or about 5 millimeters when the balloon 730 is configured in the compressed configuration. For example, in some embodiments the circumference 758 is 3 millimeters or less such as 2.90 millimeters, among other possible values that are less than 3 millimeters.

In some embodiments, the balloon 730 can have an open central region or open center 775 which has an absence of material when the balloon 730 is in the compressed configuration, as illustrated in FIG. 7B. For instance, the open center 775 can extend in part between a distal surface of the portion of the balloon 730 that forms the valley at the proximal end 732 of the balloon 730 and a proximal surface of the portion of the balloon 730 the forms the valley at the distal end 738 of the balloon 730, as illustrated in FIG. 7B. The presence of the open center 775 of the balloon 730 can promote aspects herein such as permitting one or more components or fluid to readily pass through the balloon 730 when the balloon 730 is in the folded compressed configuration.

FIG. 7C illustrates the conformable fillable balloon 730 in an expanded configuration. Upon filler material injection (e.g., hydrogel injection), the conformable fillable balloon 730 expands from the folded compressed delivery configuration, as illustrated in FIG. 7B, to an expanded deployed or inflated configuration. When configured in the expanded deployed configuration the conformable fillable balloon 730 can have an overall predetermined rectangular shape. As illustrated in FIG. 7C, the conformable fillable balloon 730 has a substantially rectangular shape with an absence of any peaks and valleys along the entire perimeter of the balloon 730. A longitudinal length 745 of the conformable fillable balloon 730 can be greater than lateral width 744 of the conformable fillable balloon 730. This rectangular configuration can yield optimal spacing with controlled dimensions (approximately a length 745 of about 18.30 millimeters and a lateral width 744 of 8.00 millimeters as shown in FIG. 7C). These dimensions are merely an example and other dimensions of the balloon 730 are possible.

In some embodiments, the balloon 730 may be distally advanced in the folded configuration using the systems described herein until the balloon 730 extends distally beyond the distal end of the sheath and is positioned at the target location. The filler material may then be transferred to the interior cavity of the balloon 730, as described herein. In some cases, the filler material may be injected through the lumen of the hypotube in a flowable state. A port 734 of the balloon 730 may be fluidly coupled to the shaft lumen to allow the filler material to be injected into the cavity of the balloon. As mentioned, the port 734 can be a one-way port such as a one-way port. As the filler material fills the cavity, the balloon 730 expands in a distal direction and in lateral directions away from the central axis of the balloon 730. The balloon 730 contains the filler material and bounds the spread of the filler material 730 as the filler material 730 gels or hardens. In some embodiments, the filler material of the present disclosure can be imaged during or after administration using a suitable imaging technique such as ultrasound or an X-ray-based imaging technique, such as computerized tomography or X-ray fluoroscopy.

Once the balloon 730 is full, the sheath and the balloon delivery system, as described herein, may be removed from the body. It is contemplated that the balloon 730 may be automatically disengaged from the balloon delivery system once the balloon 730 is full. In other embodiments, the sheath may be distally advanced to push the balloon 730 off of the balloon delivery system. In yet other examples, the balloon delivery system may be proximally retracted prior to proximal retraction of the sheath. The distal end of the sheath may act as a mechanical stop to disengage the balloon 730 from the balloon delivery system. The balloon delivery system and the sheath may be proximally retracted simultaneously, one after the other, or combinations thereof, as described herein.

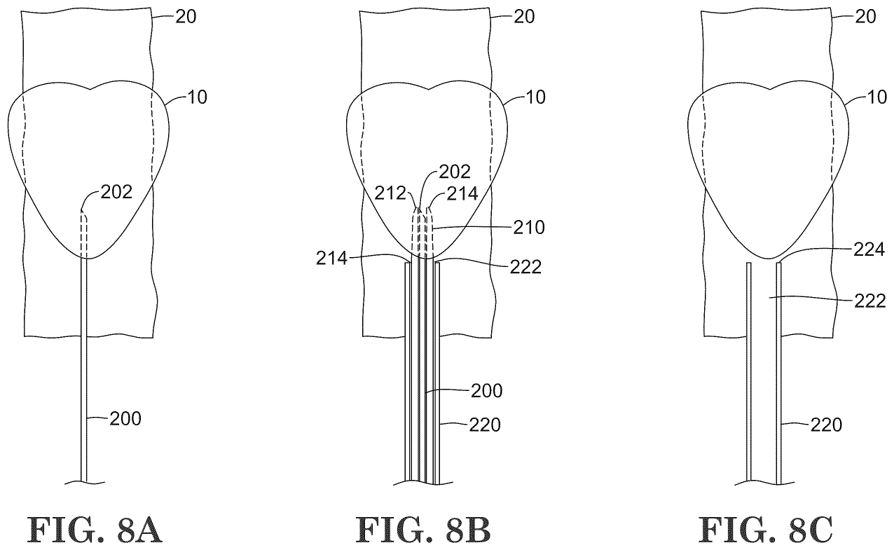

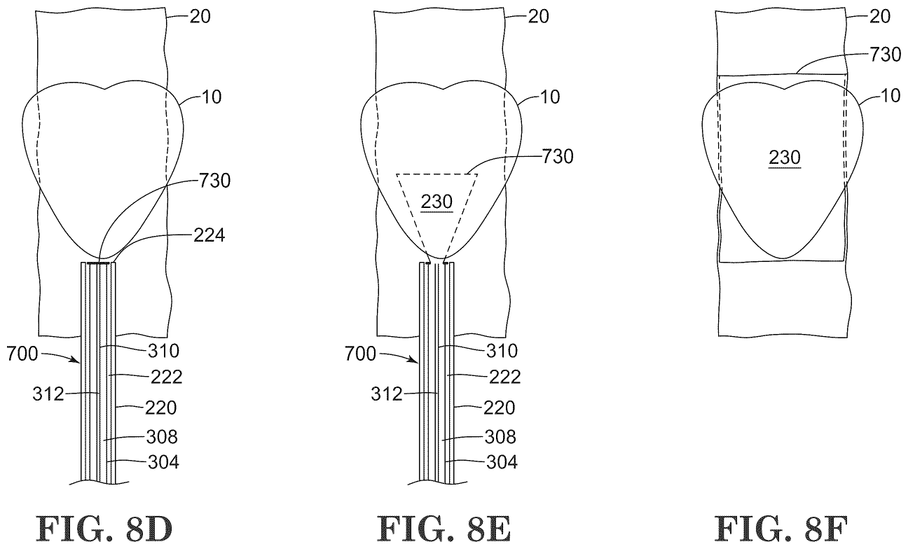

For instance, FIGS. 8A-8E schematically illustrate a method of implanting the balloon 730 in a subject. The illustrative method is shown and described with respect to implantation between the prostate 10 and the rectum 20. However, the balloon 730 may be delivered to other anatomies using similar delivery systems and methods. While certain steps are shown as a sequence between each figure, in other embodiments fewer steps are contemplated and the order by which steps are performed can be different than what is illustrated.

In some embodiments, the methods herein can comprise puncturing the intended anatomy using a dilator and advancing the dilator and sheath assembly inside the anatomy until the sheath is at an intended location in vivo (e.g., under prostate). Optionally, the methods herein can include performing dissection of tissue (e.g., hydro dissection as needed by injecting a fluid (e.g., saline) through a luer provided on dilator proximal end), retracting the dilator from the sheath, inserting and advancing a balloon assembly in vivo until the balloon reaches a distal end of the sheath. The methods herein can include retracting the sheath to expose a folded balloon, such as those described herein. The methods herein can include injecting a filler (e.g., hydrogel) until the balloon is configured in an inflated configuration (e.g., completely inflated), pulling a balloon catheter while keeping the sheath pressed against the inflated balloon until the balloon catheter assembly is detached from balloon, and/or retracting the sheath and the balloon catheter assembly from the anatomy, as described herein.

For instance, as shown in FIG. 8A, a needle 200 may be inserted to the target location. In the illustrated example, the target location may be between the prostate 10 and the rectum 20. The target location may be centrally located in the lateral direction. The needle 200 may be an 18-gauge hypodermic needle. However, the needle 200 may be smaller than 18 gauge or greater than 18-gauge, as desired. Exemplary materials for the needle include, but are not limited to, metals and metal alloys, such as stainless steel and Nitinol, and polymers. The distal tip 202 of the needle may be sharpened and may have a beveled shape. Next, a dilator 210 may be advanced over the needle 200 to widen the access path, as shown in FIG. 8B. For example, the dilator 210 may include a lumen 212 extending from a proximal end (not shown) to a distal end 214 of the dilator 210. The lumen 212 may be sized and shaped to receive the needle 200 therein. A sheath 220 may be advanced over the dilator 210. In some cases, the sheath 220 may be advanced substantially simultaneously with the dilator 210. In other cases, the access sheath 220 may be advanced over the dilator 210 after the dilator 210 has been positioned. The sheath 220 may include a lumen 222 extending from a proximal end (not shown) to a distal end 224 of the sheath 220. The lumen 222 may be sized and shaped to receive the dilator 210 and the balloon delivery system 700 therein. In some examples, the lumen 222 may have diameter of approximately 10 millimeters (mm). However, the diameter of the lumen 222 may be less than 10 mm or greater than 10 mm.

The dilator 210 may then be removed while the sheath 220 is left in place, as shown in FIG. 8C. Next the balloon delivery system 700 may be advanced through the lumen 222 of the sheath 220 with the balloon 730 in the compressed or compact configuration. The balloon delivery system 700 may be distally advanced until the balloon 730 is positioned substantially at or extends distally beyond the distal end 224 of the sheath 220 and is positioned at the target location, as shown in FIG. 8D. The filler material 230 may then be transferred to the interior cavity of the balloon 730. In some cases, the filler material 230 may be injected through the lumen 112 of the hypotube 110 in a flowable state. The port 734 of the balloon 730 may be fluidly coupled to the lumen 112 to allow the filler material 230 to be injected into the cavity of the balloon 730.

As the filler material 230 fills the cavity, the balloon 730 expands in longitudinal and/or lateral a direction away from the central axis 46 of the balloon 730. The balloon 730 contains the filler material and bounds the spread of the filler material 230 as the filler material 230 gels or hardens, as shown in FIG. 8E. In some embodiments, the filler material 230 of the present disclosure can be imaged during or after administration using a suitable imaging technique such as ultrasound or an X-ray-based imaging technique, such as computerized tomography or X-ray fluoroscopy.

Once the balloon 730 is full, the sheath 220 and the balloon delivery system 700 may be removed from the body, as shown in FIG. 8F. It is contemplated that the balloon 730 may be automatically disengaged from the balloon delivery system 700 once the balloon 730 is full. In other embodiments, the sheath 220 may be distally advanced to push the balloon 730 off of the balloon delivery system 700. In yet other examples, the balloon delivery system 700 may be proximally retracted prior to proximal retraction of the sheath 220. The distal end 224 of the sheath 220 may act as a mechanical stop to disengage the balloon 730 from the balloon delivery system 700. The balloon delivery system 7100 and the sheath 220 may be proximally retracted simultaneously, one after the other, or combinations thereof.

Next the balloon delivery system 700 may be advanced through the lumen 222 of the sheath 220 with the balloon 730 in the compressed or compact configuration. The balloon delivery system 700 may be distally advanced until the balloon 730 is adjacent to the distal end 224 of the sheath 220, as shown in FIG. 8A. However, this is not required. In some embodiments, the balloon delivery system 700 may be distally advanced until the balloon 730 extends distally beyond the distal end 224 of the sheath 220 and is positioned at the target location. The filler material 230 may then be transferred to the interior cavity of the balloon 730. In some cases, the filler material 230 may be injected through the lumen 312 of the hypotube in a flowable state. The port 734 of the balloon 330 may be fluidly coupled to the shaft lumen 308 to allow the filler material 230 to be injected into the cavity of the balloon 330.

As the filler material 230 fills the cavity, the balloon 730 expands in a distal direction and in lateral directions away from the central axis 346 of the balloon 730, as shown in FIG. 8B. The balloon 730 contains the filler material and bounds the spread of the filler material 230 as the filler material 230 gels or hardens. In some embodiments, the filler material 230 of the present disclosure can be imaged during or after administration using a suitable imaging technique such as ultrasound or an X-ray-based imaging technique, such as computerized tomography or X-ray fluoroscopy.

Once the balloon 730 is full, the sheath 220 and the balloon delivery system 700 may be removed from the body, as shown in FIG. 8C. It is contemplated that the balloon 730 may be automatically disengaged from the balloon delivery system 700 once the balloon 730 is full. In other embodiments, the sheath 220 may be distally advanced to push the balloon 730 off of the balloon delivery system 700. In yet other examples, the balloon delivery system 700 may be proximally retracted prior to proximal retraction of the sheath 220. The distal end 224 of the sheath 220 may act as a mechanical stop to disengage the balloon 730 from the balloon delivery system 700. The balloon delivery system 700 and the sheath 220 may be proximally retracted simultaneously, one after the other, or combinations thereof.

In some embodiments, one or more components of the balloon delivery system can include a luer adapter or other component at a proximal end thereof. The luer adaptor can permit introduction of a fluid such as saline and/or a filler material. For example, the luer adaptor can be permit the introduction of saline or another fluid for hydro dissection of tissue, in some embodiments.

In some embodiments, one or more components of the balloon delivery system can include markings such as marker bands disposed on a portion of a surface (e.g., exterior surface) thereof. The presence of the markings such as marker bands can aid in delivery of the components of the balloon delivery system and/or the conformable, fillable balloons 30, 330, 730, herein, to a target site or location in vivo. For instance, in some embodiments a distal tip or needle of dilator can include markings configured in an echogenic pattern or other pattern (e.g., longitudinally) along a portion of a length of the distal tip or need of the dilator.

Conformable, fillable balloons 30, 330, 730 in accordance with the present disclosure may be provided in a variety of shapes and dimensions depending on the target implantation site for the balloon. Balloon shapes include spheroidal balloon shapes, including spheres, various elongated balloon shapes including cylindrical balloon shapes which may have, for example, partial spheroidal ends (e.g., sausage-shaped balloons) or cone-shaped ends or prolate spheroids (e.g. football-shaped balloons), pear-shaped balloons, torus-shaped balloons (e.g., doughnut-shaped balloons), rectangular or square shapes, flattened balloon shapes including oblate spheroids (e.g., balloons shaped like lentils or M&M'S® candies) and other disk shaped balloons, among others.

Conformable, fillable balloons 30, 330, 730 in accordance with the present disclosure may in some embodiments include slots, apertures, or other types of orifices extending therethrough. For instance, conformable, fillable balloons 30, 330, 730, may include one or more wherein the balloon includes drainage holes for at least a portion of the filler material (e.g., hydrogel) to egress from a cavity within the filled conformable, fillable balloons 30, 330, 730.

Conformable, fillable balloons 30, 330, 730 in accordance with the present disclosure include conformable, fillable balloons 30, 330, 730 having a longest dimension (e.g., diameter for a sphere, the width for a disk, length for an elongated balloon such as a sausage-shaped balloon, etc.) ranging anywhere from 1 millimeter (mm) or less to 40 mm or more, for example, ranging from 1 mm to 2.5 mm to 5 mm to 10 mm to 20 mm to 40 mm, among other possibilities, when filled.

Conformable, fillable balloons 30, 330, 730 in accordance with the present disclosure include conformable, fillable balloons 30, 330, 730 having a volume ranging anywhere from 0.1 milliliters (ml) or less to 250 ml or more, for example, ranging anywhere from 0.1 ml to 0.2 ml to 0.5 ml to 1.0 ml to 2.5 ml to 5 ml to 10 ml to 25 ml to 50 ml to 100 ml to 250 ml, among other possibilities, when filled.

Conformable, fillable balloons 30, 330, 730 in accordance with the present disclosure include conformable, fillable balloons 30, 330, 730 having a wall thickness ranging anywhere from 10 or less micrometers to 5000 micrometers or more, for example, ranging from 10 micrometers to 25 micrometers to 50 micrometers to 100 micrometers to 250 micrometers to 500 micrometers to 1000 micrometers to 5000, among other possibilities, when filled.

Conformable, fillable balloons in accordance with the present disclosure are formed from a variety of materials, depending on the target implantation site of the balloon, including biostable balloon materials, which remain intact and retain the filler material for at least 20 years after implantation, and degradable materials which are configured to break down and be removed from the body after a time period ranging from 1 day to 24 months (e.g., ranging anywhere from 1 day to 3 days to 1 week to 2 weeks to 1 month to 3 months to 6 months to 12 months to 24 months). Degradation may occur due to various mechanisms that include hydrolysis by interaction with water in the body, enzymatic degradation by enzymes that are naturally occurring in the body, oxidation by oxidants produced by the body and/or degradation brought about by substances such as enzymes that are introduced into the body.

Balloon materials for use herein include biostable polymeric materials and degradable polymeric materials, including biodegradable polymeric materials. Some illustrative balloon materials are described in U.S. Patent Application Ser. No. 63/673,063 filed on Jul. 18, 2024 and titled MATERIALS FOR CONFORMABLE FILLABLE MEDICAL BALLOONS, the disclosure of which is hereby incorporated by reference. Degradable polymeric materials may undergo bond cleavage, resulting in reduced molecular weight and solubilization of the smaller polymer chains in biological fluids. The dissolved polymer chains may be completely metabolized, excreted by the kidneys, and/or absorbed via other physiological mechanisms.

Balloon materials for use herein are generally biocompatible with the space in which they are implanted. In some embodiments, the balloon materials are compliant (stretchable) materials such as those that comprise elastomeric polymers. In some embodiments, the balloon materials are thermoplastic polymers. In some embodiments, the balloon materials are in the form of crosslinked networks, including hydrogel crosslinked networks.

Biostable polymeric materials may be selected, for example, from the following polymers, among others: polyamides, polyolefins including polypropylene, polybutadienes including hydrogenated polybutadienes, polydimethylsiloxane (PDMS), polyurethanes, styrene block copolymers, including styrene-isobutylene-styrene triblock copolymers, styrene-isoprene-styrene triblock copolymers, styrene-butadiene-styrene triblock copolymers, and styrene-isoprene/butadiene-styrene triblock copolymers (e.g., Kraton™ polymers from Kraton Corporation, The Woodlands, TX, USA).

Degradable polymeric materials may be selected, for example, from the following polymers, among others: polyesters including polylactones such as polyvalerolactone and poly(l-lactide-co-ε-caprolactone), polycarbonates such as poly(hexamethylene carbonate) (polyHMC), polydioxane, poly(lactide co-glycolide), polyethylene glycol, polyvinyl alcohol, water-soluble polyacrylates (e.g. polyacrylic acid), polyoxazolines, polypeptides and proteins.

Degradable polymeric materials may be selected, for example, from hydrophilic polymer hydrogels that comprises crosslinks between polymer chains within the hydrophilic polymer hydrogel. The polymer chains are generally hydrophilic polymer chains that are soluble in water and can be cleared from the body, for example, by metabolic processes, clearance through the kidneys, and/or other physiological processes.

As used herein, a “hydrogel” is a crosslinked polymer that contains water or can absorb water but does not dissolve when placed in water, although hydrogels may degrade in vivo over time or be degraded by introduction of ex vivo substances.

When desired, cleavage of the hydrophilic polymer hydrogel that forms the balloon can initiated by contacting the hydrophilic polymer hydrogel compositions with a cleavage composition.

If the filler material 230 is a biostable or a biodegradable solid, the filler material 230 may remain, at least for a time, after the polymeric material forming the balloon degrades. If the filler material 230 is a biodegradable solid, the filler material 230 ultimately biodegrades.

A variety of filler materials 230 may be used in conjunction with conformable, fillable balloons 30, 330, 730 of the present disclosure, including biostable and degradable filler materials.

In some embodiments the filler materials 230 may be in the form of aqueous liquids including aqueous solutions and aqueous dispersions, particular examples of which include normal saline, phosphate buffered saline, 5% dextrose in water (D5W), contrast media including radiographic contrast media such as iodinated contrast media (e.g., Lipiodol® Guerbet LLC, Princeton, NJ, USA), iohexol (Omnipaque™, GE HealthCare Technologies, Inc., Chicago, IL, USA), iodixanol (Visipaque™, GE HealthCare Technologies, Inc.), iopamidol, ioversol, etc.) and barium sulfate, magnetic resonance imaging (MRI) contrast media including gadolinium-containing contrast media, near-infrared (NIR) contrast media such as those containing indocyanine green, methylene blue, sodium fluorescein, and 5-aminolevulinic acid (5-ALA), and positron emission tomography (PET) contrast media such as those containing [¹⁸F]fluorodeoxyglucose (FDG) or [¹⁸F]sodium fluoride (Na¹⁸F).

In some embodiments the filler materials 230 may be in the form of a polymeric solutions, for example, solutions of a water-soluble polymer selected from polysaccharides (e.g., solutions of hyaluronic acid, gelatin, pectin, alginate, cellulose, gellan gum, etc.), polyethylene glycol, polyoxazolines, polypeptides, polyacrylates, polyacrylamides, copolymers of polyethylene glycol, and polyvinyl alcohol.

In some embodiments the filler materials 230 may be in the form of a shear thinning hydrogel, which is able to temporarily fluidize under shear stress and recover its original mechanical properties after release of the applied stress, based on natural polymers such as gelatin (e.g., Obsidio™ Conformable Embolic from Boston Scientific, a pre-hydrated bioresorbable mixture of gelatin and layered silicate particles, either with or without tantalum powder for radiocontrast), collagen, hyaluronic acid, alginate, and chitosan. Such hydrogels are generally crosslinked based on physical crosslinking mechanisms such as electrostatic interactions or hydrogen bonding.

In some embodiments the filler materials 230 may be in the form of a lower critical solution temperature (LCST) material, where the LCST refers to the temperature at which the transition from a liquid phase to a hydrogel phase occurs. When the LCST material is injected into a body, the temperature of the LCST material is below the LCST and increases due to heat transfer from the body. Once the temperature of the LCST material reaches the LCST, the transition from the liquid phase to the hydrogel phase takes place. Such LCST materials can be configured or prepared to remain in a liquid phase, with a low viscosity, at an injection temperature below the body temperature (e.g., room temperature or below) and to transform to a gel phase when increased in temperature to body temperature. Examples of such materials include polyoxyethylene-polyoxyproplyene (PEO-PPO) block copolymers, such as Pluronic acid F127 and F108, and N-Isopropyl acrylamide (IPAAm) copolymers.

In some embodiments the filler materials 230 may form crosslinked hydrogels in vivo. An example of such a crosslinked hydrogel is SpaceOAR®, which is based on a multi-arm polyethylene glycol (PEG) polymer functionalized with succinimidyl glutarate as activated end groups which further react with trilysine to form crosslinks. During use, a solution of the multi-arm polymer and the lysine is simultaneously injected with a buffer solution. When mixed, the buffer solution increases the pH and dramatically accelerates the rate of reaction between the multi-arm polymer and the lysine, forming a crosslinked hydrogel within seconds. Another example of such a crosslinked hydrogel is SpaceOAR Vue®, which, like SpaceOAR®, is based on a multi-arm polyethylene glycol (PEG) polymer functionalized with succinimidyl glutarate as activated end groups which further react with trilysine to form crosslinks. In SpaceOAR Vue®, some of the succinimidyl glutarate end groups are functionalized with 2,3,5-triiiodobenzamide groups, providing radiopacity. In other embodiments, systems of these types can be used to form hydrogels ex vivo, after which the hydrogels are broken down into particles and suspended in an aqueous solution to form an injectable pre-formed hydrogel.

In some embodiments the filler materials 230 may comprise a hydrophilic polymer hydrogel that comprises crosslinks between hydrophilic polymer chains within the hydrophilic polymer hydrogels, which crosslinks contain hydrolysable linkers, as described above.

In some embodiments the filler materials 230 may comprise a hydrophilic polymer hydrogel that comprises crosslinks between hydrophilic polymer chains within the hydrophilic polymer hydrogels, which crosslinks contain immolative linkers that are dispersed throughout the hydrogels, as described above. Such hydrogels can be degraded in situ by contact with a suitable cleavage composition, as described above.

Filler materials 230 for use in the present disclosure may also contain one or more additional agents such as therapeutic agents, imaging agents, colorants, tonicity adjusting agents, and pH adjusting agents. In embodiments where the balloon permits release (e.g., by allowing diffusion through the balloon), such agents (e.g., therapeutic agents, imaging agents, etc.) may be released from the balloon over time.

Examples of therapeutic agents include antithrombotic agents, anticoagulant agents, antiplatelet agents, thrombolytic agents, antiproliferative agents, anti-inflammatory agents, hyperplasia inhibiting agents, anti-restenosis agent, smooth muscle cell inhibitors, antibiotics, antimicrobials, analgesics, anesthetics, growth factors, growth factor inhibitors, cell adhesion inhibitors, cell adhesion promoters, anti-angiogenic agents, cytotoxic agents, chemotherapeutic agents, checkpoint inhibitors, immune modulatory cytokines, T-cell agonists, STING (stimulator of interferon genes) agonists, antimetabolites, alkylating agents, microtubule inhibitors, hormones, hormone antagonists, monoclonal antibodies, antimitotics, immunosuppressive agents, tyrosine and serine/threonine kinases, proteasome inhibitors, mRNA, matrix metalloproteinase inhibitors, Bcl-2 inhibitors, DNA alkylating agents, spindle poisons, poly (DP-ribose)polymerase (PARP) inhibitors, and combinations thereof.

Examples of imaging agents include (a) fluorescent dyes such as fluorescein, indocyanine green, or fluorescent proteins (e.g. green, blue, cyan fluorescent proteins), (b) contrast agents for use in conjunction with magnetic resonance imaging (MRI), including contrast agents that contain elements that form paramagnetic ions, such as Gd(III), Mn(II), Fe(III) and compounds (including chelates) containing the same, such as gadolinium ion chelated with diethylenetriaminepentaacetic acid, (c) contrast agents for use in conjunction with ultrasound imaging, including organic and inorganic echogenic particles (i.e., particles that result in an increase in the reflected ultrasonic energy) or organic and inorganic echolucent particles (i.e., particles that result in a decrease in the reflected ultrasonic energy), (d) contrast agents for use in connection with near-infrared (NIR) imaging, which can be selected to impart near-infrared fluorescence to the hydrogels of the present disclosure, allowing for deep tissue imaging and device marking, for instance, NIR-sensitive nanoparticles such as gold nanoshells, carbon nanotubes (e.g., nanotubes derivatized with hydroxy or carboxyl groups, for instance, partially oxidized carbon nanotubes), dye-containing nanoparticles, such as dye-doped nanofibers and dye-encapsulating nanoparticles, and semiconductor quantum dots, among others, and NIR-sensitive dyes such as cyanine dyes, squaraines, phthalocyanines, porphyrin derivatives and boron dipyrromethane (BODIPY) analogs, among others, (e) imageable radioisotopes including 99mTc, 201Th, 51Cr, 67Ga, 68Ga, 111In, 64Cu, 89Zr, 59Fe, 42K, 82Rb, 24Na, 45Ti, 44Sc, 51Cr and 177Lu, among others, and (f) radiocontrast agents, for example, particles of tantalum, tungsten, rhenium, niobium, molybdenum, and their alloys, which metallic particles may be spherical or non-spherical. Additional examples of radiocontrast agents include non-ionic radiocontrast agents, such as iohexol, iodixanol, ioversol, iopamidol, ioxilan, or iopromide, ionic radiocontrast agents such as diatrizoate, iothalamate, metrizoate, or ioxaglate, and iodinated oils, including ethiodized poppyseed oil (available as Lipiodol®).

Examples of colorants include brilliant blue (e.g., Brilliant Blue FCF, also known as FD&C Blue 1), indigo carmine (also known as FD&C Blue 2), indigo carmine lake, FD&C Blue 1 lake, and methylene blue (also known as methylthioninium chloride), among others.

Examples of tonicity adjusting agents include sugars (e.g., dextrose, lactose, etc.), polyhydric alcohols (e.g., glycerol, propylene glycol, mannitol, sorbitol, etc.) and inorganic salts (e.g., potassium chloride, sodium chloride, etc.), among others.

Examples of pH adjusting agents include various buffer solutes.

In other aspects, the present disclosure provides balloon implantation kits that include one or more inflatable balloons 30, 330, 730 configured to be implanted in a human body, one or more balloon delivery catheters 100, 300, 700, and one or more containers that include a filler material 230 that is configured to be introduced into the one or more balloons 30, 330, 730 or that include filler material precursors that, when combined, produce a filler material in the one or more balloons. Containers for the filler material or the filler material precursors include, for example, vials, ampules, empty syringes, and preloaded syringes. In various embodiments, the one or more inflatable balloons further include features for preventing outflow of filler material such as check valves, plugs or constricting devices as described above.

In some embodiments, the balloon implantation kits include one or more filling tubes (e.g., catheter tubes or hypotubes) for introducing the filler material or filler material precursors into the one or more balloons. In some embodiments, the one or more filling tubes are releasably attached to the one or more balloons.

In some embodiments, the balloon implantation kits include one or more delivery sheaths 220 through which the one or more balloons is/are implanted in the subject. In some of these embodiments, the one or more balloons is/are pre-loaded in the one or more delivery sheaths.

In some embodiments, the balloon implantation kits include one or more containers that contain a cleavage solution. Containers for the cleavage solution include, for example, vials, ampules, and preloaded syringes. In these embodiments, the balloon implantation kits may further include a catheter tube for delivering the cleavage solution. In some embodiments, a distribution head, such as a spray head, may be disposed at a distal end of the catheter tube.

In some embodiments, the balloon implantation kits further include sterile packaging in which the above-described the kit components are removably packaged in a sterile state.

What is claimed is:

1. A balloon delivery and balloon system comprising:
an elongate shaft extending from a proximal end to a distal end;
a first lumen extending from the proximal end to the distal end of the elongate shaft;
a hypotube extending from a proximal end to a distal end;
a second lumen extending from the proximal end to the distal end of the hypotube; and
a conformable, fillable balloon releasably coupled to the distal end of the hypotube, the conformable, fillable balloon defining a cavity in fluid communication with the second lumen of the hypotube;
wherein the conformable, fillable balloon is expandable from a compressed lower profile delivery configuration to an expanded deployed configuration;
wherein the conformable, fillable balloon is configured to contain a filler material.

2. The system of claim 1, wherein the conformable, fillable balloon comprises a port at a proximal end thereof.

3. The system of claim 2, wherein the port is disposed within the first lumen of the elongate shaft.

4. The system of claim 2, wherein the port is disposed over and around an outer surface of the hypotube.

5. The system of claim 2, wherein the port comprises a one-way valve.

6. The system of claim 1, wherein the conformable, fillable balloon is compressed such that a maximum lateral width of the balloon is less than an inner diameter of an access sheath.

7. The system of claim 1, wherein the conformable, fillable balloon has a series of undulating peaks and valleys when configured in a manufactured configuration.

8. The system of claim 7, wherein the peaks and valleys are spaced a distance apart.

9. The system of claim 8, wherein the peaks and valleys each are a respective first height.

10. The system of claim 9, wherein the conformed, fillable balloon is compressed longitudinally to a compressed configuration such that a compressed length of the conformable, fillable balloon in the compressed configuration is less than a length of the conformable, fillable balloon in the expanded deployed configuration.

11. The system of claim 10, wherein:
the conformable, fillable balloon configured in the compressed configuration has a substantially circular shape; and
the peaks and valleys of the conformable, fillable balloon configured in the compressed configuration each are a respective second height that is greater than the respective first height.

12. The system of claim 11, wherein the conformable, fillable balloon configured in the compressed configuration is configured to form a substantially rectangular shape when configured in the expanded deployed configuration.

13. A method of implanting a conformable, fillable balloon in a body, the method comprising:
inserting a needle to a target location in the body;
advancing a dilator over the needle to widen an access path;
advancing a sheath over the dilator;
removing the dilator while leaving the sheath in place;
advancing a balloon delivery system through a lumen of the sheath, with a conformable, fillable balloon in a compressed configuration releasably coupled to the balloon delivery system;
positioning the balloon at the target location;
transferring a filler material to an interior cavity of the balloon, causing the balloon to expand to an expanded and deployed configuration; and
removing the sheath and the balloon delivery system from the body.

14. The method of claim 13, wherein transferring the filler material comprises injecting the filler material through a lumen of the balloon delivery system.

15. The method of claim 13, further comprising imaging the filler material during or after administration using ultrasound or an X-ray-based imaging technique.

16. The method of claim 13, wherein the conformed, fillable balloon is compressed longitudinally.

17. The method of claim 16, wherein the conformable, fillable balloon has a series of undulating peaks and valleys.

18. The method of claim 17, wherein the conformable, fillable balloon in the compressed configuration has substantially circular shape.

19. The method of claim 17, wherein the conformable, fillable balloon in the expanded and deployed configuration has a substantially rectangular shape with an absence of any peaks or valleys.

20. A kit for delivering a conformable, fillable balloon to a target location in a body, the kit comprising:
an access sheath;
a balloon delivery system comprising:
an elongate shaft extending from a proximal end to a distal end and defining a first lumen extending from the proximal end to the distal end of the elongate shaft;
a hypotube extending from a proximal end to a distal end and defining a second lumen extending from the proximal end to the distal end of the hypotube, the hypotube configured to be received in the first lumen of the elongate shaft;
a conformable, fillable balloon releasably coupled adjacent to the distal end of the elongate shaft, the conformable, fillable balloon expandable from a compressed lower profile delivery configuration to an expanded deployed configuration;
wherein an interior cavity of the balloon is in fluid communication with the lumen of the elongate shaft; and
wherein the conformable, fillable balloon is configured to contain a filler material and bound a spread of the filler material as the filler material gels or hardens.
